(19) United States
(12) Patent Application Publication
(10) Pub. No.: US 2017/0095660 A1
(43) Pub. Date: Apr. 6, 2017

(54) IONTOPHORESIS INJECTION DEVICE AND INJECTION METHOD

(71) Applicant: K-HEALTHWEAR CO., Ltd., Daejeon (KR)

(72) Inventors: Hoi Jun YOO, Daejeon (KR); Ki Seok SONG, Seoul (KR)

(73) Assignee: K-HEALTHWEAR CO., Ltd., Daejeon (KR)

(21) Appl. No.: 15/128,109

(22) PCT Filed: Apr. 29, 2014
(86) PCT No.: PCT/KR2014/003758

§ 371 Date: Sep. 22, 2016

(51) Int. Cl. A61N 1/32; A61N 1/04; A61N 1/08; A61M 37/00; A61N 1/02

(52) U.S. Cl. CPC A61N 1/325; A61M 37/00; A61N 1/025; A61N 1/08; A61N 1/0476; A61N 1/0448; A61M 2037/0007; A61M 2205/3317; A61M 2230/65; A61M 2205/3584; A61M 2205/3561; A61M 2205/3368; A61M 2205/52; A61M 2205/502

(57) ABSTRACT

An iontophoretic drug delivery apparatus comprises an electrode unit comprising a plurality of iontophoresis electrodes and a plurality of tissue resistivity measurement electrodes; a programmable current unit configured to control a current that is supplied to the iontophoresis electrodes to thereby control the amount of drug delivered; an impedance detection unit comprising a detection mode that selectively measures a load resistance value between the iontophoresis electrodes or a tissue resistivity value between the tissue resistivity measurement electrodes so as to monitor the amount of drug delivered; and a control unit configured to control the programmable current unit by determining the amount of drug delivered or whether the drug is to be delivered, based on the load resistance or tissue resistivity value measured in the impedance detection unit.

### TECHNICAL FIELD

The present invention relates to an iontophoretic drug delivery apparatus and drug delivery method, which comprise measuring the load resistance and tissue resistivity values of a subject to thereby monitor a drug delivery state and control drug delivery.

### BACKGROUND ART

Methods of delivering drugs to the human body include iontophoresis in which electric current is applied to an ionized drug so that the drug is introduced into the human body.

Specifically, in iontophoresis, electric current is applied to the skin so that a charged drug can be delivered into the skin by electrical repulsion without causing pain, whereby the skin can be physiologically improved by the drug.

Iontophoresis uses a pair of electrodes to apply electric current to the skin. When a certain amount of electric current I is applied to electrodes for a certain time t, a drug in a suitable amount corresponding to the charge quantity Q=I×t is delivered into the skin of a subject.

Meanwhile, the load resistance of an iontophoresis apparatus is defined as a resistance value that determines the current value measured by applying a certain voltage to both ends of an electrode coming in contact with the skin of a subject.

The load resistance R of the subject is divided into the contact resistance R_{CONT} between the electrode and the skin and the tissue resistance R_{TIS}, and is expressed as R=(2×R_{CONT})+R_{TIS}.

Generally, R_{CONT} is a few KOhms, whereas R_{TIS} is only a few tens Ohms which is about 100 times lower than R_{CONT}. For this reason, the load resistance is measured using two electrodes, most of the component is R_{CONT}, and the R_{TIS} component is negligible, and thus it appears that the load resistance is equal to the contact resistance between the electrode and the skin.

Meanwhile, in the case in which the voltage V between electrodes is constant, given that the load resistance is R, the value of current that is introduced into the skin is determined according to the load resistance value, based on the Ohm's law I=V/R.

Thus, when the load resistance value of the iontophoresis apparatus is changed, the amount of electric current supplied is also changed, and the amount of charge that is supplied to the skin in proportion to the current is also changed, and thus the instantaneous amount of drug that is delivered is also changed.

Meanwhile, when a drug is continuously delivered to the skin of a subject so that the cumulative amount of drug delivered increases, the resistivity of the skin decreases. Thus, the amount of drug delivered can be determined by measuring the resistivity of the skin.

For reference, an example of a measurement device based on iontophoresis is disclosed in Korean Patent No. 10-0730582 (published on Jun. 20, 2007; entitled “Iontophoresis apparatus”).

The conventional iontophoresis apparatus comprises: a plurality of electrodes included in a mask or patch to be attached to the skin of a user; and an iontophoresis chip module electrically connected to the electrodes; the iontophoresis chip module comprising: a wireless rechargeable unit able to be charged in a non-contact charging manner; a microprocessor operating by the power received from the wireless rechargeable unit and storing a control program; a control drive for controlling voltage, frequency and current applied to the electrodes in response to a command of the microprocessor; an output unit connected to the control drive to transmit a certain current to the electrodes; a skin diagnosis measuring unit connected to the output unit and receiving bio-impedance of the user measured from the electrodes; and an A/D converter for converting analog data detected from the skin diagnosis measuring unit into digital data and inputting the converted digital data into the microprocessor.

This conventional iontophoresis apparatus can measure the resistance of the skin of a subject by applying electric current to the skin before treatment, determine the optimal voltage, current and frequency suitable for the skin condition of the subject based on the measured load resistance value, and supply a certain current to the skin through the electrodes. However, it has a problem in that, because it cannot measure the load resistance value that changes during an iontophoresis procedure, it cannot stably deliver a drug according to the condition of the subject during delivery of the drug.

In addition, the conventional iontophoresis apparatus makes it possible to measure the skin condition of a subject before treatment, but has a problem in that it cannot measure the amount of drug accumulated in the skin during an iontophoresis procedure.

### DISCLOSURE

### Technical Problem

The present invention has been made in order to solve the above-described problems, and it is an object of the present invention to provide an iontophoretic drug delivery apparatus and drug delivery method, in which the contact state of electrodes, human skin conditions and the amount of drug delivered are monitored in real time during an iontophoresis procedure, and the amount of drug to be delivered and whether the drug is to be delivered are determined based on the monitored information.

### Technical Solution

To achieve the above object, an iontophoretic drug delivery apparatus and drug delivery method according to an embodiment of the present invention comprise: an electrode unit comprising a plurality of iontophoresis electrodes and a plurality of tissue resistivity measurement electrodes; a programmable current unit configured to control a current that is supplied to the iontophoresis electrodes to thereby control the amount of drug delivered; an impedance detection unit comprising a detection mode that selectively measures a load resistance value between the iontophoresis electrodes or a tissue resistivity value between the tissue resistivity measurement electrodes so as to monitor the amount of drug delivered; and a control unit configured to control the programmable current unit by determining the amount of drug delivered or whether the drug is to be delivered, based on the load resistance or tissue resistivity value measured in the impedance detection unit.

The apparatus may comprise a wireless communication unit configured to convert information of the control unit to a wireless signal and communicate the converted wireless signal with an external device in a wireless manner.

The impedance detection unit may comprise: an alternating current generating unit configured to supply alternating current to the iontophoresis electrodes; and a voltage sensor unit configured to measure a voltage that is generated in the iontophoresis electrodes and the tissue resistivity measurement electrodes.

The detection mode of the impedance detection unit is an instantaneous drug delivery amount-detecting mode that measures the load resistance value, and in this case, the control unit supplies alternating current to the iontophoresis electrodes in a state in which operation of the programmable current unit is stopped, and the control unit may control the programmable control unit based on the measured load resistance value between the iontophoresis electrodes.

The detection mode of the impedance detection mode is a cumulative drug amount-detecting mode that measures the tissue resistivity value, and in this case, the control unit supplies alternating current to the iontophoresis electrodes in a state in which operation of the programmable current unit is stopped, and the control unit may control the programmable current unit based on the measured tissue resistivity value between the tissue resistivity measurement electrodes so that the drug may be delivered.

The detection mode of the impedance detection unit may comprise different detection modes that are sequentially selected and operated for a predetermined time.

The control unit may be configured to control one or more values selected from among the amplitude, frequency and duty cycle values of the current that is supplied from the programmable current unit to the iontophoresis electrodes.

The external device may comprise a display that communicates with the wireless communication unit in a wireless manner to observe information about the subject.

The external device may comprise an input means that inputs drug delivery information so as to control the control unit by communication with the wireless communication unit.

The electrode unit may comprise an electrode switching unit that, according to the detection mode, selectively connects the iontophoresis electrodes and the tissue resistivity measurement electrodes to the impedance detection unit or selectively changes the polarity of the current that is transferred from the programmable current unit to the iontophoresis electrodes.

The electrode unit may be configured such that a pair of the tissue resistivity measurement electrodes disposed between a pair of the iontophoresis electrodes, or a pair of the iontophoresis electrodes disposed between a pair of the tissue resistivity measurement electrodes.

The electrode unit may be configured such that the plurality of iontophoresis electrodes and the plurality of tissue resistivity measurement electrodes, which are alternately disposed to be spaced apart from each other, and either the tissue resistivity measurement electrodes disposed between the iontophoresis electrodes, or the iontophoresis electrodes disposed between the tissue resistivity measurement electrodes, may be provided in pairs.

The electrode unit may be configured such that the iontophoresis electrodes and the tissue resistivity measurement electrodes are radially disposed in concentric circles having different diameters.

The electrode unit may further comprise a temperature sensor unit for measuring the temperature of a portion to which the drug is delivered, and the temperature signal may also be transmitted to the external device.

The control unit may stop operation of the programmable current unit to stop delivery of the drug, when the temperature measured in the temperature sensor unit is out of a predetermined temperature range input in the control unit.

The apparatus may comprise a drug-containing drug pad that is detachably coupled to the iontophoresis electrodes.

The drug pad may comprise a portion formed of a porous material so that the drug is impregnated into the portion.

The drug pad may comprise an adhesive layer provided on one or both surfaces of the drug pad.

The drug pad may comprise a drug information memory unit configured to store information about the drug and provide the information to the control unit.

An iontophoretic drug delivery method using an iontophoretic drug delivery apparatus comprising a plurality of iontophoresis electrodes and a plurality of tissue resistivity measurement electrodes comprises: a first monitoring step of supplying alternating current to the iontophoresis electrodes to thereby monitor the delivery state of the drug based on a measured load resistance value between the iontophoresis electrodes; a second monitoring step of supplying alternating current to the iontophoresis electrodes to thereby monitor the delivery state of the drug based on a measured tissue resistivity value between the tissue resistivity measurement electrodes; and a step of controlling delivery of the drug based on the load resistance value or tissue resistivity value measured in the first monitoring step and the second monitoring step.

The iontophoretic drug delivery apparatus may further comprise a temperature measurement sensor for measuring the temperature of a portion to which the drug is delivered, the step of controlling delivery of the drug may further comprise a step of stopping the delivery of the drug by blocking supply of the current to the iontophoresis electrodes when the temperature measured in the temperature measurement sensor is out of a predetermined temperature range.

The first monitoring step may comprise blocking the current that is supplied to the iontophoresis electrodes, and supplying a low-frequency alternating current to the iontophoresis electrode, and sensing a voltage between the iontophoresis electrodes to thereby measure the load resistance value.

The second monitoring step may comprise blocking the current that is supplied to the iontophoresis electrodes, and supplying a high-frequency alternating current to the iontophoresis electrode.

The first monitoring step or the second monitoring step may further comprise a step of blocking the current that is supplied to the iontophoresis electrodes when a current load resistance value or tissue resistivity value is out of a predetermined ideal value range, and sensing the abnormal state or end-state of drug delivery through a warning means.

When the current load resistance value or tissue resistivity value in the first monitoring step or the second monitoring step is out of the predetermined ideal value range, one or more values selected from among the amplitude, frequency and duty cycle values of the current that is supplied to the iontophoresis electrodes may be controlled.

The first monitoring step or the second monitoring step may comprise controlling one or more values selected from among the amplitude, frequency and duty cycle values of the current that is supplied to the iontophoresis electrodes, when the current load resistance value or tissue resistivity value is within the predetermined range.

An iontophoretic drug delivery apparatus comprises: an electrode unit comprising a plurality of iontophoresis electrodes and a plurality of tissue resistivity measurement electrodes spaced apart from the iontophoresis electrodes; a programmable current unit configured to control a current that is supplied to the iontophoresis electrodes to thereby control the amount of drug delivered; a programmable current unit configured to control the amplitude of a current that is supplied to the iontophoresis electrodes to thereby control the amount of drug delivered; an impedance detection unit configured to selectively measure either a load resistance between the iontophoresis electrodes, obtained after supplying the current between the iontophoresis electrodes and sensing a voltage, generated due to the load resistance between the iontophoresis electrodes, by the iontophoresis electrodes, or a tissue resistivity, obtained after supplying the current between the iontophoresis electrodes and sensing a voltage, generated due to the tissue resistivity between the tissue resistivity measurement electrodes, by the tissue resistivity measurement electrodes; a control unit configured to control the programmable current unit by determining the amount of drug delivered or whether the drug is to be delivered, based on the load resistance or tissue resistivity value measured in the impedance detection unit; and a wireless communication unit configured to convert information of the control unit to a wireless signal and communicate the converted wireless signal with an external device.

The present invention also provides an iontophoretic drug delivery apparatus comprising a plurality of iontophoresis electrodes and a programmable current unit configured to control one or more values selected from among the amplitude, frequency and duty cycle values of a current that is supplied to the iontophoresis electrodes to thereby control the amount of drug delivered, the apparatus being configured to: supply current to the iontophoresis electrodes coming into contact with a body portion; sense a voltage, generated due to tissue load of the applied current, by tissue resistivity measurement electrodes physically spaced apart from the iontophoresis electrodes, thereby measuring a tissue resistivity; and monitor the cumulative amount of drug delivered, based on the tissue resistivity, thereby controlling the programmable current.

### Advantageous Effects

The iontophoretic drug delivery apparatus according to the present invention comprises a plurality of electrodes, and can accurately monitor and control the amount of drug delivered during an iontophoresis procedure by measuring a load resistance value and a tissue resistivity value through the impedance detection unit.

Specifically, the control unit measures the instantaneous amount of drug delivered to the human skin, based on the load resistance value measured by the impedance detection unit. When the instantaneous amount of drug delivered changes, the control unit controls the programmable current unit so that the instantaneous amount of drug delivered into the skin is maintained at a constant level, whereby a constant amount of the drug can be stably delivered into the skin.

In addition, the control unit measures the cumulative amount of drug delivered into the skin, based on the tissue resistivity value measured by the impedance detection unit, and control unit controls the programmable current unit based on the cumulative amount of drug delivered, whereby a constant amount of the drug can be stably delivered into the skin.

Furthermore, when a current load resistance value decreases or a change in the load resistance value frequently occurs, the control unit determines that the contact state between the human skin and the electrode unit is abnormal, and controls the programmable current unit so that the supply of current from the programmable current unit is stopped, whereby a constant amount of the drug can be stably delivered into the skin.

Moreover, in order to prevent the subject's skin from getting burned by heating of the electrode unit due to the load resistance between the electrodes, the control unit may control the programmable control unit so as to stop the supply of current from the programmable current unit, when a body temperature value measured by the temperature sensor unit is out of a predetermined temperature range input in the control unit.

In addition, the wireless communication unit transmits the state information of a subject to an external device, it is possible to check the iontophoresis procedure state of the subject through the external device, and the iontophoresis procedure can be remote-controlled through the external device.

### DESCRIPTION OF DRAWINGS

FIG. 1 is a black diagram showing an iontophoretic drug delivery apparatus according to an embodiment of the present invention.

FIGS. 2 to 6 are top views showing the electrode unit of an iontophoretic drug delivery apparatus according to an embodiment of the present invention.

FIG. 7 is a top view showing a state in which the electrodes of an electrode unit in an iontophoretic drug delivery apparatus according to an embodiment of the present invention are connected in a drug delivery mode among detection modes.

FIG. 8 is a schematic view showing a state in which the electrodes of an electrode unit in an iontophoretic drug delivery apparatus according to an embodiment of the present invention are connected in an instantaneous drug delivery amount-detecting mode among detection modes.

FIG. 9 is a schematic view showing a state in which the electrodes of an electrode unit in an iontophoretic drug delivery apparatus according to an embodiment of the present invention are connected in an instantaneous drug delivery amount-detecting mode among detection modes.

FIG. 10 is a top view showing a drug pad in an iontophoretic drug delivery apparatus according to an embodiment of the present invention.

FIG. 11 is a side cross-sectional view showing a drug pad in an iontophoretic drug delivery apparatus according to an embodiment of the present invention.

FIG. 12 is a side cross-sectional view showing another example of a drug pad in an iontophoretic drug delivery apparatus according to an embodiment of the present invention.

FIG. 13 is a flow chart showing an iontophoretic drug delivery method according to an embodiment of the present invention.

FIG. 14 is a flow chart showing a step of controlling drug delivery based on a load resistance value in the first monitoring step of an iontophoretic drug delivery method according to an embodiment of the present invention.

FIG. 15 is a flow chart showing a step of controlling drug delivery based on a tissue resistivity value in the second monitoring step of an iontophoretic drug delivery method according to an embodiment of the present invention.

### DESCRIPTION OF REFERENCE NUMERALS

100: electrode unit; 110: iontophoresis electrodes;

120: tissue resistivity measurement electrodes; 200: electrode switching unit;

150: drug pad; 155: adhesive layer;

300: programmable current unit; 400: impedance detection unit;

410: alternating current generating unit; 420: voltage sensor unit;

500: temperature sensor unit; 600: control unit;

700: wireless communication unit; 800: external device;

900: subject's skin.

### MODE FOR INVENTION

Hereinafter, embodiments of the present invention will be described with reference to the accompanying drawings.

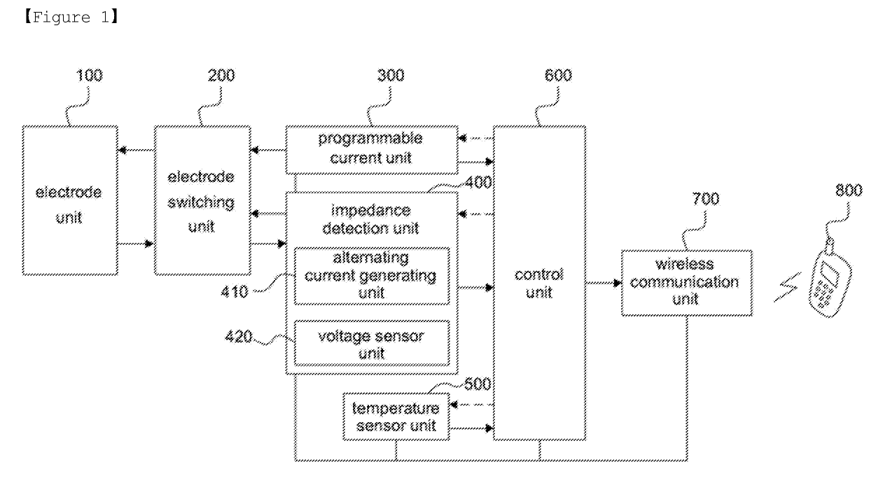

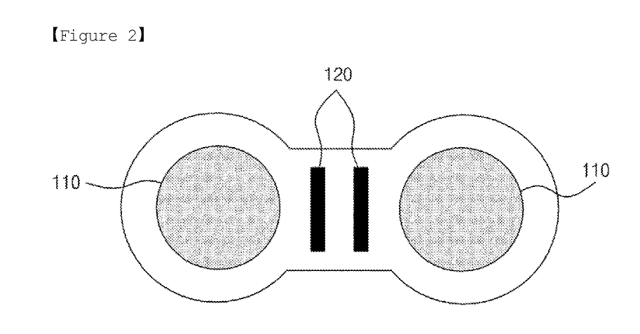

As shown in FIGS. 1 and 2, an iontophoretic drug delivery apparatus according to an embodiment of the present invention may comprise an electrode unit 100.

The electrode unit 100 may include a plurality of electrodes configured to come into contact with a subject's skin 900 and to apply electric current to the skin.

Meanwhile, the electrode unit 100 may comprise iontophoresis electrodes 110 and tissue resistivity measurement electrodes 120.

The iontophoresis electrodes 110 are a pair of spaced electrodes configured to come into contact with the subject's skin 900 so that electric current supplied from a programmable current unit 300 to be described below will be applied to the skin in order to deliver a drug into the skin.

Herein, the iontophoresis electrodes 110 may include one or more pairs of a plurality of electrodes.

In addition, the iontophoresis electrodes 110 may be configured to supply a low-frequency or high-frequency alternating current to a subject according to a sensing mode selected from an impedance detection unit 400 to be described below.

A pair of the tissue resistivity measurement electrodes 120 may be disposed to be spaced apart from each other between a pair of iontophoresis electrodes 110 so that they will measure a voltage, which is produced by a current generated from the iontophoresis electrodes 110, through a subject's skin 900, when an impedance detection unit 400 to be described below measures the tissue resistivity value of the subject's skin.

Herein, the tissue resistivity measurement electrodes 120 may include one or more pairs of a plurality of electrodes.

Furthermore, the electrode unit 100 may comprise an electrode switching unit 200.

The electrode switching unit 200 may be configured to selectively the iontophoresis electrodes 110 and the tissue resistivity measurement electrodes 120 to the impedance detection unit 400 in order either to supply electric current to deliver a drug into the subject's skin 900 or to enable the impedance detection unit 400 to measure a load resistance or tissue resistivity value.

For example, when the impedance detection unit 400 measures the load resistance between the iontophoresis electrodes 100, the electrode switching unit 200 connects the alternating current generating unit 410 of the impedance detection unit 400 to the iontophoresis electrodes 110 so as to supply a low-frequency alternating current to the iontophoresis electrodes 100, and connects the iontophoresis electrodes 110 to the voltage sensor unit 420 of the impedance detection unit 400 so that the voltage sensor unit 420 specifies the voltage between the iontophoresis electrodes 110.

In addition, when the impedance detection unit 400 measures a tissue resistivity value, the electrode switching unit 200 connects the alternating current generating unit 410 of the impedance detection unit 400 to the iontophoresis electrodes 110 so as to supply a high-frequency alternating current between the iontophoresis electrodes 110, and connects the voltage sensor unit 420 to the tissue resistivity measurement electrodes 120 so that the voltage sensor unit 420 of the impedance detection unit 400 senses the voltage between the tissue resistivity measurement electrodes 120.

Herein, the electrode switching unit 200 may selectively change one or more value of the amplitude, cycle and duty cycle of current.

Moreover, when current is supplied to the iontophoresis electrode 110 to deliver a drug, the electrode switching unit 200 connects the iontophoresis electrodes 110 to the programmable current unit 300 so as to enable current to be stably supplied to the iontophoresis electrode 110, and can selectively change the polarity of current that is transferred to the iontophoresis electrodes 110.

Meanwhile, the electrode unit 100 may further comprise a temperature sensor unit 500.

In order to prevent a subject from getting burned by heat generated in the electrode unit 100 due to the load resistance between the iontophoresis electrodes 110 or drug side effects when the drug is delivered, the temperature sensor unit 500 may measure the temperature of a subject's skin portion to which the drug is delivered, and provide the measured temperature value to a control unit 600 to be described below.

As shown in FIGS. 2 to 6, the electrode unit 100 may be configured in various forms so as to come into contact with the subject's skin 900 to apply current to the skin.

Herein, the amount of drug delivered can be controlled according to the size of the electrode unit 100.

For example, as the area of the electrode unit 100 increases, the drug delivery rate and the amount of drug delivered can increase, and as the area of the electrode unit 100 decreases, the drug delivery rate and the amount of drug delivered can decrease.

As shown in FIG. 2, the electrode unit 100 may be configured such that a pair of tissue resistivity measurement electrodes 120 are disposed between a pair of iontophoresis electrodes 110 or a pair of iontophoresis electrodes 110 are disposed between a pair of tissue resistivity measurement electrodes 120.

As shown in FIGS. 3 and 4, the electrode unit 100 may be configured such that a plurality of iontophoresis electrodes 110 and a plurality of tissue resistivity measurement electrodes 120 are alternately disposed to be spaced apart from each other. Herein, the tissue resistivity measurement electrodes 120 disposed between the iontophoresis electrodes 110, or the iontophoresis electrodes 110 disposed between the tissue resistivity measurement electrodes 120 may be provided in pairs.

As shown in FIGS. 5 and 6, the electrode unit 100 may be configured such that a plurality of iontophoresis electrodes 110 and a plurality of tissue resistivity measurement electrodes 120 are radially disposed in concentric circles having different diameters.

As shown in FIG. 1, the iontophoretic drug delivery apparatus according to the embodiment of the present invention may comprise a programmable current unit 300.

The programmable current unit 300 may apply current to the iontophoresis electrodes 110 to deliver a drug to a subject's skin 900, and may control the amplitude, frequency and duty cycle of current which is supplied to the iontophoresis electrodes 110, thereby controlling the amount of drug delivered or the instantaneous amount of drug delivered.

Herein, the current which is supplied from the programmable current unit 300 to the iontophoresis electrodes 110 may have one or more selected from among certain amplitude, frequency, and duty cycle values.

In addition, the current which is supplied from the programmable current unit 300 to the iontophoresis electrodes 100 may be direct current or sinusoidal or square-wave alternating current.

As shown in FIG. 1, the iontophoretic drug delivery apparatus according to the embodiment of the present invention may comprise an impedance detection unit 400.

The impedance detection unit 400 has a detection mode that selectively measures the load resistance value between the iontophoresis electrodes 110 or the tissue resistivity value between the tissue resistivity measurement electrodes 120 so as to enable a drug to be stably delivered in an amount input in a control unit 600 to be described below, and it can transmit the measured value to the control unit 600.

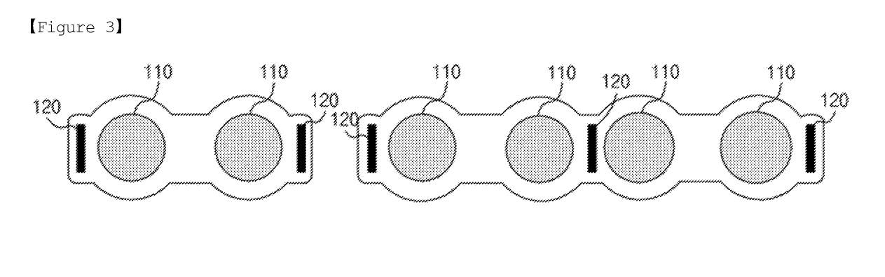

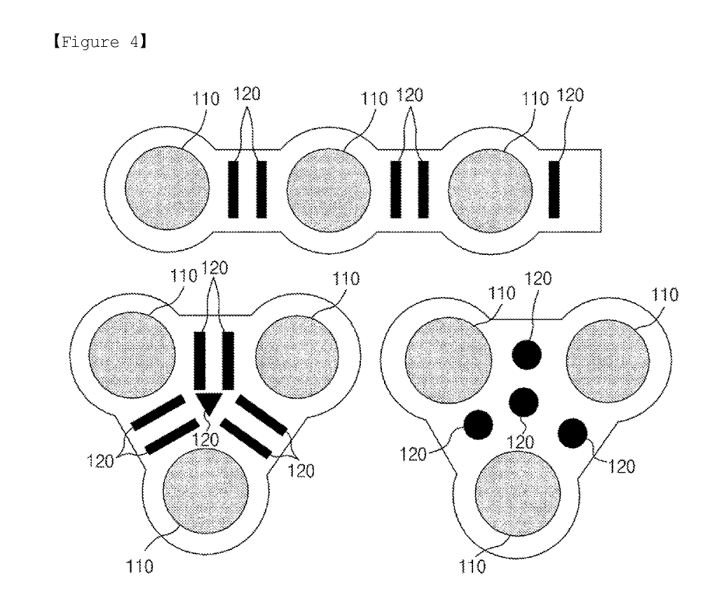

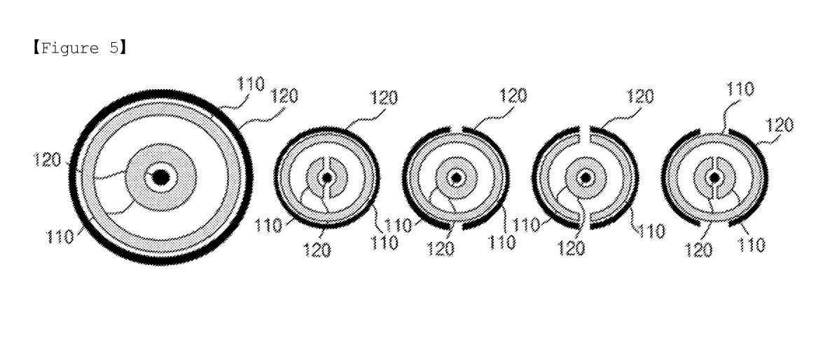

As shown in FIGS. 7 to 9, the detection mode of the impedance detection unit 400 may comprise a drug delivery mode, an instantaneous drug delivery amount-detecting mode and a cumulative drug delivery amount-detecting mode.

The instantaneous drug delivery amount-detecting mode measures the load resistance value between the iontophoresis electrodes 110 in order to measure the instantaneous amount of drug delivered, and the cumulative drug delivery amount-detecting mode measures the tissue resistivity value between the tissue resistivity measurement electrodes 120 so as to enable the impedance detection unit 400 to measure the cumulative amount of drug delivered into the skin.

The drug delivery mode supplies current to the iontophoresis electrodes 120 so as to deliver a drug to the skin.

The impedance detection mode 400 may be configured such that the detection modes, specifically the instantaneous drug delivery amount-detecting mode, the cumulative drug delivery amount-detecting mode and the drug delivery mode, can be sequentially selected and operated by the control unit 600 for a predetermined time in order to monitor the amount of drug stably delivered into the subject's skin 900 during drug delivery.

Meanwhile, the impedance detection unit 400 may comprise an alternating current generating unit 410 and a voltage sensor unit 420.

The alternating current generating unit 410 comprises an oscillator that generates alternating current to be supplied to the iontophoresis electrodes 110. In the instantaneous drug delivery amount-detecting mode, the alternating current generating unit 410 supplies low-frequency alternating current between the iontophoresis electrodes 110, and in the cumulative drug delivery amount-detecting mode, the alternating current generating unit 410 supplies high-frequency alternating current to the iontophoresis electrodes 110.

In addition, the voltage sensor unit 420 comprises a voltage sensor that senses a voltage to measure the voltage value of the electrode unit 100. When the detection mode is the instantaneous drug delivery amount-detecting mode, the voltage sensor unit 420 measures the voltage value between the iontophoresis electrodes 110, and when the detection mode is the cumulative drug delivery amount-detecting mode, the voltage sensor unit 420 measures the voltage value between the tissue resistivity measurement electrodes 120.

When the detection mode of the impedance detection unit 400 is the instantaneous drug delivery amount-detecting mode, the alternating current generating unit 410 supplies low-frequency alternating current to the iontophoresis electrodes 110, and the voltage sensor unit 420 measures the voltage between the iontophoresis electrodes 110 to thereby measure the load resistance value.

In addition, when the detection mode of the impedance detection unit 400 is the cumulative drug delivery amount-detecting mode, the alternating current generating unit 410 supplies high-frequency alternating current to the iontophoresis electrodes 110, and the voltage sensor unit 420 measures the voltage between the tissue resistivity measurement voltages 120 to thereby measure the tissue resistivity value.

As shown in FIG. 1, the iontophoretic drug delivery apparatus according to the embodiment of the present invention may comprise a control unit 600.

The control unit 600 can select a predetermined detection mode from the impedance detection unit 400, and control the programmable current unit 300 based on a load resistance value and tissue resistivity value measured according to the selected detection mode.

Specifically, when the control unit 600 selects the drug delivery mode, the control unit 600 can control the programmable current unit 300 to supply current having certain amplitude, frequency and duty cycle values to the iontophoresis electrodes 110 so as to deliver a drug to a subject.

Herein, the current that is supplied from the programmable current unit 300 to the iontophoresis electrodes 110 may have one or more values selected from among certain amplitude, frequency and duty cycle values.

Herein, the programmable current unit 300 can determine a current value according to a program pre-stored in the programmable current unit so as to deliver the drug to the subject.

Herein, the pre-stored program may be a program that determines a current value from the amount of drug delivery calculated according to any equation based on the kind of drug, the condition of the subject, the amount of the drug and the drug delivery time.

This program is known technology and can be modified in various manners by application of various parameters, and thus the detailed description thereof is omitted.

When the control unit 600 selects the instantaneous drug delivery amount-detecting mode, the control unit 600 blocks current supply from the programmable current unit 300, and controls the impedance detection unit 400 so that the impedance detection unit 400 determines the load resistance value between the iontophoresis electrodes 110.

When the initially calculated load resistance value input in the control unit 600 is compared with the current load resistance value and the current load resistance value is greater or smaller than the predetermined reference value, it is determined that the instantaneous amount of drug administered decreases or increases, and thus the programmable current unit 300 is controlled to change the value of current that is supplied to the iontophoresis electrodes 110 so that the initially calculated load resistance value can be maintained.

In addition, when the current load resistance value is out of the predetermined range of the initially calculated load resistance value, the control unit 600 determines that drug delivery is abnormal, and thus blocks the supply of current from the programmable current unit 300 to the iontophoresis electrodes 110.

When the control unit 600 selects the cumulative drug delivery amount-detecting mode, the control unit 600 controls the programmable unit 300 so as to block current supply from the programmable current unit 300, and controls the impedance detection unit 400 so that the impedance detection unit 400 calculates the tissue resistivity value between the tissue resistivity measurement electrodes 120.

Furthermore, the control unit 600 determines a target tissue resistivity value according to a conversion equation that coverts the drug delivery amount input in the control unit 600 into a tissue resistivity value. When the target tissue resistivity value and the current tissue resistivity value are compared with each other and the current tissue resistivity value is greater than a predetermined reference value compared to the target tissue resistivity value, the control unit 600 determines that the current amount of drug accumulated in the skin is lower than the drug delivery amount input in the control unit 600, and thus the control unit 600 controls the programmable current unit 300 so as to maintain or increase the value of current that is supplied from the programmable current unit 300 to the iontophoresis electrodes.

In addition, when the current tissue resistivity value is equal to or smaller than the target tissue resistivity value, the control unit 600 determines that the current amount of drug accumulated in the skin is equal to or greater than the drug delivery amount input in the control unit 600, and thus controls the programmable current unit 300 so as to block the current that is supplied from the programmable current unit 300 to the iontophoresis electrodes 110.

Meanwhile, in order to prevent the subject's skin 900 from getting burned due to an increase in the temperature of the electrodes, when the electrode temperature value provided by the temperature sensor unit 500 is out of the predetermined temperature range input in the control unit 600, the control unit 600 blocks the current that is supplied from the programmable current unit 300 to the iontophoresis electrodes 110.

As shown in FIG. 1, the iontophoretic drug delivery apparatus according to the embodiment of the present invention may include a wireless communication unit 700.

The wireless communication unit 700 may be configured to convert the iontophoresis procedure state information obtained from the control unit 600 into a wireless signal, and communicate the wireless signal with an external device 800 in a wireless manner so as to send the iontophoresis procedure state information of the subject through the external device 800.

Herein, the iontophoresis procedure state information may be current amplitude, frequency, duty cycle, load resistance value, the subject's skin temperature, the instantaneous amount of drug delivered, the cumulative amount of drug delivered, and the delivery time.

In addition, although the wireless communication unit 700 is described, it is to be understood that the wireless communication unit 700 can be connected to the external device 800 by wire so as to communicate with the external device by wire.

The external device 800 may comprise a display that visually shows the iontophoresis procedure state information sent from the wireless communication unit 700, and thus can inform an observer of the iontophoresis procedure state information in various forms such as letters, figures, graphs and images.

Particularly, when the control unit 600 determines that the iontophoresis procedure state information is abnormal, it give the observer a warning of the abnormality through the external device 800.

In addition, the external device 800 may comprise an input unit that inputs drug delivery information, including the kind of drug, the amount of drug delivered, and the drug delivery time, so that the wireless communication unit 700 can receive the drug delivery information input in the input unit, and based on the received drug delivery information, the control unit 600 can control the programmable current unit 300 so that the kind of drug, the amount of drug delivered and the drug delivery time can be controlled by the external device.

For example, the external application 800 may include an application capable of controlling the iontophoresis apparatus so that the application can receive and transmit drug delivery information, including the kind of drug, the amount of drug delivered and the drug delivery time, and the wireless communication unit 700 can receive the transmitted information, and the control unit 600 can control the programmable current unit 300 based on the received information.

Herein, it is to be understood that the application installed in the external device 800 can receive iontophoresis procedure state information and inform the observer of the information in various forms, including letters, figures, graphs and images.

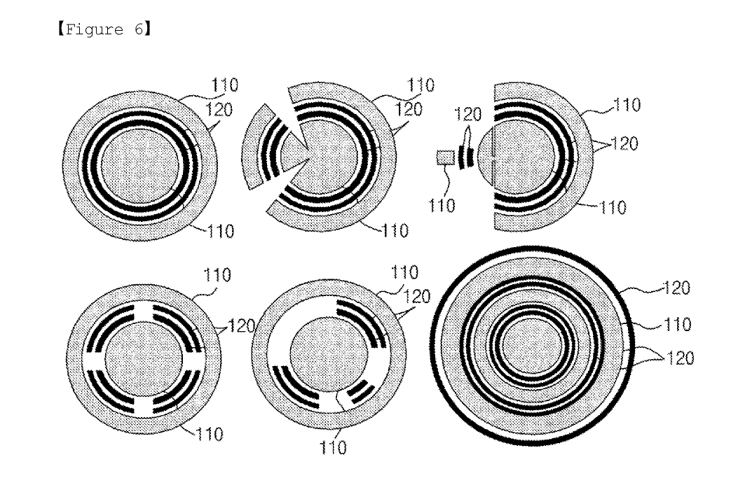

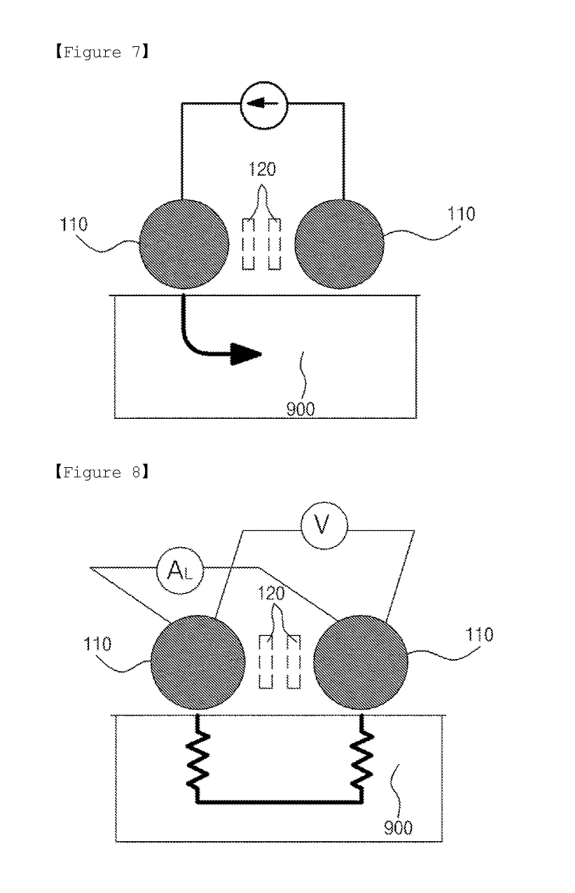

As shown in FIGS. 10 and 11, the iontophoretic drug delivery apparatus according to the embodiment of the present invention may comprise a drug pad 150.

The drug pad 150 may contain a drug to be delivered, and a portion or all of the drug pad 150 may be formed of a porous material so that it can be impregnated with the drug.

Herein, a portion 151 of the drug pad 150, which is formed of a porous material and impregnated with the drug, can be formed to correspond to the arrangement, shape, number and the like of the iontophoresis electrodes 110.

In addition, the porous material portion 151 of the drug pad 150 may be formed of an electrically conductive material so that current can be supplied to the drug.

Meanwhile, the drug pad 150 may comprise an adhesive layer 155 so that it can be detachably coupled to the electrode unit 100, particularly iontophoresis electrodes 110, and can also be detachably attached to the subject's skin 900.

The adhesive layer 155 may be provided on one or both of a surface of the drug pad 150, which is attached to the iontophoresis electrode 110, and a surface of the drug pad 150, which is attached to the body. In addition, the adhesive layer 155 may be provided only around the portion 151 made of the porous material.

Herein, the adhesive layer 155 may comprise an electrically conductive material so that electricity can pass therethrough, like the drug pad 150.

In addition, the drug pad 150 may comprise a drug information memory unit 157. The drug information memory unit 157 may store information about the drug impregnated into the drug pad, for example, the name of the drug, the components of the drug, the side effects of the drug, a delivery method for the drug, etc.

Particularly, the drug information memory unit 157 may store a basic current value for drug delivery. Based on the current value for drug delivery, the control unit 600 can control the programmable current unit 300 to control the amount of drug to be delivered, according to a suitable current value.

Herein, it is to be understood that the drug information memory unit 157 is electrically connected to the control unit 600, when the drug pad 150 is attached to the iontophoresis electrodes 110.

In another example, a drug pad 150′ may be made of a non-porous, electrically conductive sheet, and one surface thereof, which comes into contact with the body, may comprise a drug layer 151′ made of the drug.

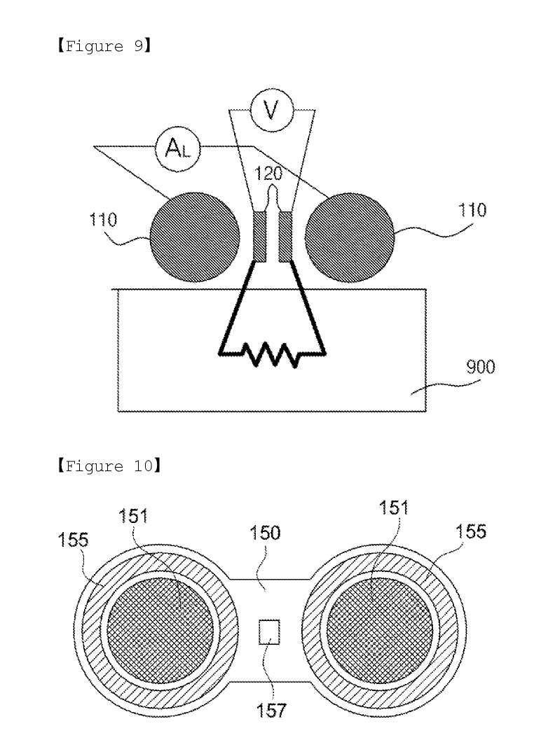

As shown in FIG. 12, the drug pad 150′ may be configured such that one surface thereof, which comprises the drug layer 151′, and the other surface coming into contact with the iontophoresis electrodes 110, include an adhesive layer 150′ which comes into contact with the iontophoresis electrodes 110 and the body. In this case, the drug pad 150′ may also include a drug information memory unit 157′ that stores drug information.

Herein, the drug layer 151′ may be provided in the electrically conductive sheet so as to correspond to the shape, arrangement, number and the like of the iontophoresis electrodes 110.

Hereinafter, the operation and effect of each of the above-described components will be described.

First, the iontophoretic drug delivery apparatus according to the embodiment of the present invention may be configured such that all the components excluding the electrode unit is provided as a single chip (SoC) and the chip is provided in the patch-type electrode unit 100.

Furthermore, the drug pad 150 impregnated with a drug is attached to the iontophoresis electrodes 110 by the adhesive layer 155, and the adhesive layer 155 provided on the opposite surface is attached to the subject's skin 900.

In the iontophoretic drug delivery apparatus according to the embodiment of the present invention, a portable power source (e.g., portable battery) for deriving the iontophoretic drug delivery apparatus may be integrally or detachably provided.

Herein, the portable battery may comprise the wireless communication unit 700.

In the iontophoretic drug delivery apparatus according to the embodiment of the present invention, the electrode unit 100 comprising the iontophoresis electrodes 110 and the tissue resistivity measurement electrodes 120 is brought into contact with the skin so that current is applied to the subject's skin 900 to deliver the drug into the skin.

Herein, the drug delivery apparatus is in a state in which the pad provided in the electrode unit 100 is impregnated with the drug or the drug pad 150 is attached to the electrode unit.

Meanwhile, the control unit 600 selects the drug delivery mode so that an externally input amount of the drug is delivered into the skin. Then, the control unit controls the programmable current unit 300 so that the programmable current unit 300 supplies a current having certain amplitude, frequency and duty cycle values to the iontophoresis electrodes 110, whereby a predetermined current is supplied to the iontophoresis electrodes 110 for a predetermined time.

Herein, the current that is supplied from the programmable current unit 300 to the iontophoresis electrodes 110 may have one or more values selected from among certain amplitude, frequency and duty cycle values.

Specifically, the programmable current unit 300 supplies a certain amount of current I to the iontophoresis electrodes 110 for a certain time t so that the drug is delivered into the subject's skin 900 in an amount corresponding to a suitable Q value determined by the charge quantity Q=I×t.

When a certain amount of current is supplied to the iontophoresis electrodes 110 as described above, the ionized drug introduced into the skin transfers ions to a freely moving electrolyte present in the human body so as to be accumulated in the skin, or flows with a freely moving liquid such as blood so as to be delivered into the subject.

Meanwhile, the control unit 600 selects the instantaneous drug delivery amount-detecting mode after a predetermined time in order to detect the change in instantaneous drug delivery amount between the iontophoresis electrodes 110.

Herein, the instantaneous drug delivery amount is equal to current I flowing between the iontophoresis electrodes 110, according to an equation expressed as the charge quantity Q=I×t.

When a voltage is constant, current I is inversely proportional to load resistance R according to the Ohm's law equation I=V/R. Thus, when the control unit 600 measures the value of load resistance R and detects a change in the value, it can detect a change in the instantaneous amount of drug delivered.

Meanwhile, load resistance R is divided into the contact resistance R_{CONT} between an electrode and the skin and the tissue resistance R_{TIS}, and is expressed as R=(2×R_{CONT})+R_{TIS}.

Generally, R_{CONT} is a few KOhms, whereas R_{TIS} is only a few tens Ohms which is about 100 times lower than R_{CONT}. For this reason, the load resistance is measured using two electrodes, most of the component is R_{CONT}, and the R_{TIS} component is negligible, and thus it appears that the load resistance is equal to the contact resistance between the electrodes and the skin, that is, the resistance value between the iontophoresis electrodes 110.

According to this relationship, when the control unit 600 selects the instantaneous drug delivery amount-detecting mode, the control unit 600 blocks the supply of current from the programmable current unit 300 in order to detect a change in the load resistance value, and controls the impedance detection unit 400 so that the impedance detection unit 400 calculates the load resistance value between the iontophoresis electrodes 110.

At this time, the alternating current generating unit 410 of the impedance detection unit 400 supplies a low-frequency alternating current of 4 Hz between the iontophoresis electrodes 110, and the voltage sensor unit 420 calculates the load resistance value between the iontophoresis electrodes 110 by measuring the voltage therebetween, and transmits the calculated load resistance value to the control unit 600.

In addition, the control unit 600 compares the current calculated load resistance value with the initially calculated load resistance value input in the control unit 600. When the current load resistance value is greater than a predetermined reference value compared to the initially calculated load resistance, the control unit 600 determines that the value of current decreases, based on the Ohm's law I=V/R, and the instantaneous amount of drug administered decreases. Thus, the control unit 600 controls the programmable unit 300 so that the value of current generated in the programmable current unit 300 increases in order to increase the instantaneous amount of drug delivered.

On the other hand, when the current calculated load resistance value is compared with the initially calculated load resistance value input in the control unit 600 and when the current load resistance value is smaller than a predetermined reference value compared to the initially calculated load resistance value, the control unit 600 determines that the value of current increases, based on the Ohm's law I=V/R, and the instantaneous amount of drug administered increases. Thus, the control unit 600 controls the programmable unit 300 so that the value of current generated in the programmable current unit 300 increases in order to decrease the instantaneous amount of drug delivered. During an iontophoresis procedure, the instantaneous amount of drug is measured by calculating the load resistance between the iontophoresis electrodes 110, and when the instantaneous amount of drug administered changes, the control unit 600 controls the programmable current unit 300 so that the instantaneous amount of amount delivered into the skin is maintained at a constant level, whereby the drug can be stably delivered into the subject's skin 900 in the amount input in the control unit 600.

In addition, when the current load resistance value increases to exceed a predetermined range or when a change in the load resistance value frequently occurs, the control unit 600 determines that the contact between the subject's skin 900 and the electrode unit 100 and the drug delivery state are poor, and controls the programmable current unit 300 so that current supply from the programmable current unit 300 is stopped, whereby the drug can be stably delivered into the subject's skin 900 in the amount input in the control unit 600.

Meanwhile, after a predetermined time after the instantaneous drug delivery amount-detecting mode, the control unit 600 selects the cumulative drug delivery amount-detecting mode in order to measure the amount of drug accumulated in the skin.

Herein, as the control unit 600 selects the drug delivery mode, the drug introduced into the skin transfers the ionized drug to a freely moving electrolyte present in the human body so as to be accumulated into the skin or flows into a freely moving liquid such as blood to move to other portions of the body.

Herein, the time-dependent increase in the drug accumulated in the skin is given as the following equation: dQ/dt=F−(Q/τ), wherein F is the flow rate of the drug introduced from the outside; Q/τ is the velocity at which the drug introduced into the blood moves to other portions; Q is the quantity of drug introduced into the subject's skin 900; and τ is a time constant.

Because of F>Q/τ for a general drug, a significant amount of the drug is accumulated between the iontophoresis electrodes 110 during the iontophoresis procedure. When the iontophoresis procedure is stopped, the accumulated drug completely disappears with the passage of time by the action of blood.

Thus, because the drug is accumulated in the skin during the iontophoresis procedure and the tissue resistivity value is decreased or increased by the amount of drug accumulated, the amount of drug accumulated can be determined by determining the tissue resistivity value.

According to this relationship, when the control unit 600 selects the cumulative drug amount-detecting mode, the control unit 600 controls the programmable current unit 300 so as to block the supply of current from the programmable current unit 300 to the iontophoresis electrode 110, and controls the impedance detection unit 400 so that the impedance detection unit 400 calculates the tissue resistivity value between the tissue resistivity measurement electrodes 120.

At this time, the alternating current generating unit 410 of the impedance detection unit 400 supplies a high-frequency alternating current of 16 Hz between the tissue resistivity measurement electrodes 120, and the voltage sensor unit 420 calculates the voltage between the iontophoresis electrodes 110 to calculate the tissue resistivity value and transmits the calculated tissue resistivity value to the control unit 600.

Furthermore, the control unit 600 determines a target tissue resistivity value according to a conversion equation that coverts the drug delivery amount input in the control unit 600 into a tissue resistivity value. When the target tissue resistivity value and the current tissue resistivity value are compared with each other and the current tissue resistivity value is greater than a predetermined reference value compared to the target tissue resistivity value, the control unit 600 determines that the current amount of drug accumulated in the skin is lower than the drug delivery amount input in the control unit 600, and thus the control unit 600 controls the programmable current unit 300 so as to maintain or increase the value of current that is supplied from the programmable current unit 300 to the iontophoresis electrodes 110.

On the other hand, when the current tissue resistivity value is equal to or lower than the target tissue resistivity value, the control unit 600 determines that the amount of drug accumulated is equal to or greater than the drug delivery amount input in the control unit 600, and controls the programmable current unit 300 so as to block the current that is supplied from the programmable current unit 300 to the iontophoresis electrodes 110, so that the iontophoresis procedure is ended.

When the amount of drug accumulated in the subject's skin 900 during the iontophoresis procedure is measured, the state of the subject's skin 900 can be determined, and the drug can be stably delivered to the subject's skin 900 in the amount input in the control unit 600.

In addition, in each detection mode, the control unit 600 compares the drug delivery amount input in the control unit 600 with the current amount of drug delivered, and when the amount of drug administered is equal to or greater than the amount of drug input, the control unit 600 controls the programmable current unit 300 so as to stop the supply of current from the programmable current unit 300, so that the iontophoresis procedure can be ended.

Meanwhile, the control unit 600 transmits the detection mode and the iontophoresis procedure state information of the subject to the wireless communication unit 700, and the wireless communication unit 700 converts the state communication to a wireless signal and transmits the converted signal to the external device 800.

Thus, the observer can observe the iontophoresis procedure state information of the subject through the display of the external device 800 even in a remote place.

In addition, through the input unit of the external device 800 and an application installed in the external device 800, the iontophoresis procedure for the subject can be remote-controlled by inputting the kind of drug, the amount of drug delivered and the drug delivery time.

Hereinafter, an iontophoretic drug delivery method using the iontophoretic drug delivery apparatus according to the embodiment of the present invention will be described.

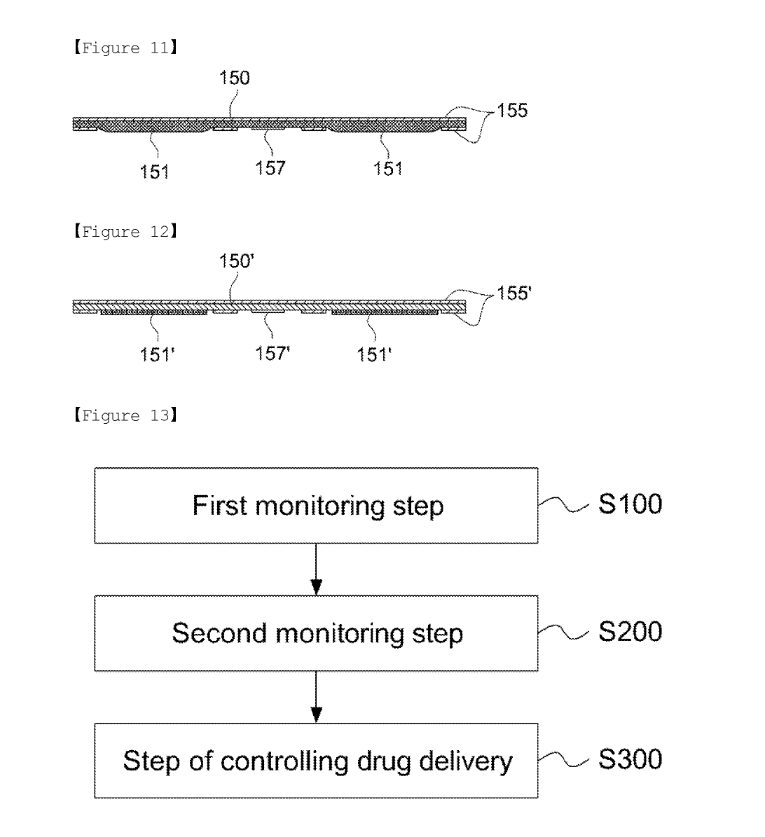

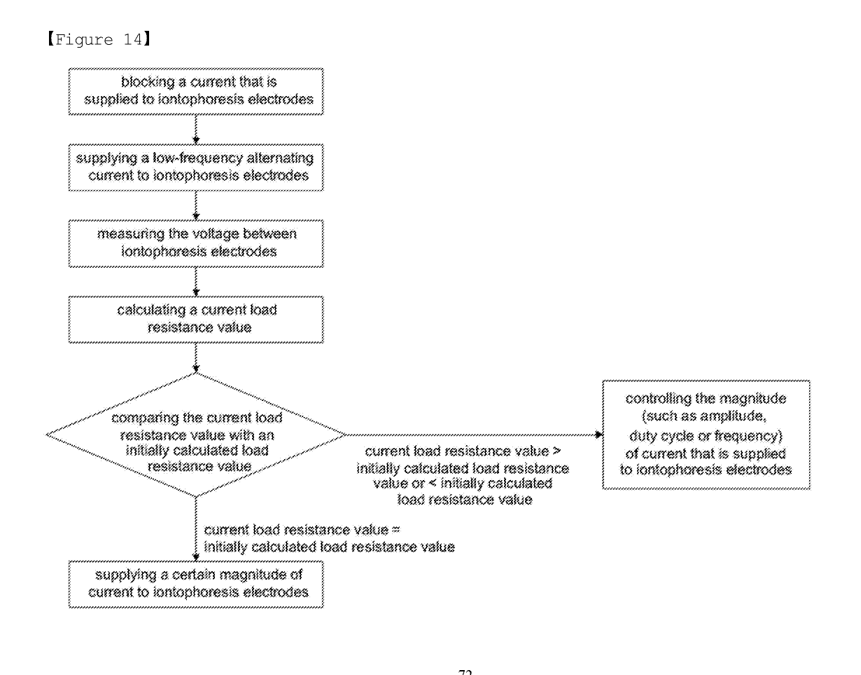

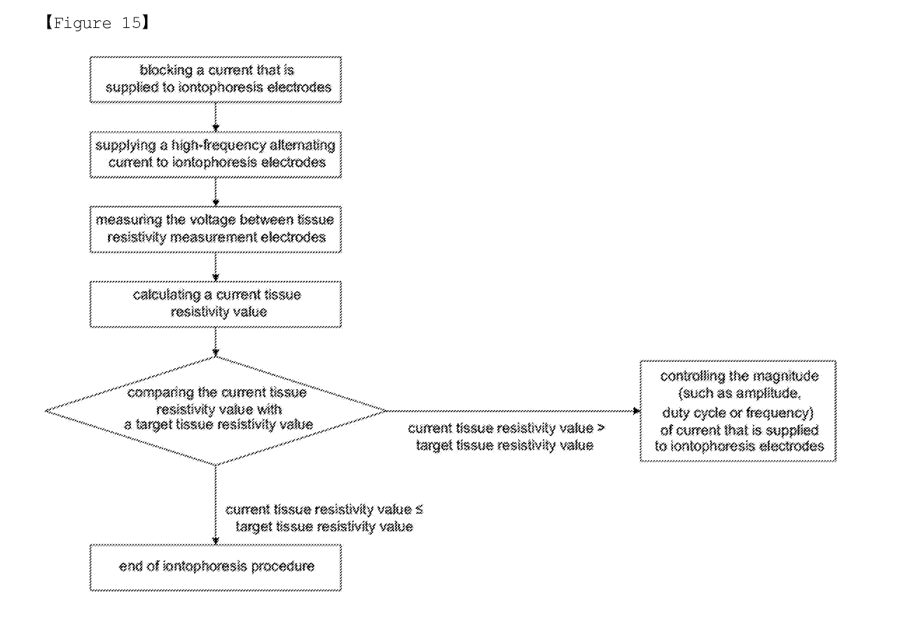

As shown in FIGS. 13 to 15, an iontophoretic drug delivery method according to the present invention may comprise a first monitoring step (S100).

The first monitoring step (S100) is a step of supplying alternating current to the iontophoresis electrodes 110 to thereby monitor the delivery state of the drug based on the measured load resistance value between the iontophoresis electrodes 110.

Specifically, the control unit 600 blocks the supply of current from the programmable current unit 300 to the iontophoresis electrodes 110 in order to detect a change in the load resistance value, and controls the impedance detection unit 400 so that the impedance detection unit 400 calculates the load resistance value between the iontophoresis electrodes 110.

At this time, the alternating current generating unit 410 of the impedance detection unit 400 supplies a low-frequency alternating current of 4 Hz between the iontophoresis electrodes 110, and the voltage sensor unit 420 senses the voltage between the iontophoresis electrodes 110 to calculate a load resistance value and transmits the calculated load resistance value to the control unit 600.

Furthermore, the control unit 600 compares the current calculated load resistance value with the initially calculated load resistance value input in the control unit 600. When the current load resistance value is greater than a predetermined reference value compared to the initially calculated load resistance value, the control unit 600 determines that the value of current decreases, according to the Ohm's law I=V/R, and the instantaneous amount of drug delivered decreases.

On the other hand, when the initially calculated load resistance value input in the control unit 600 is compared with the current calculated load resistance value and the current calculated load resistance value is lower than a predetermined reference value compared to the initially calculated load resistance value, the control unit 600 determines that the value of current increases, according to the Ohm's law I=V/R, and the instantaneous amount of drug delivered increases.

Herein, when the initially calculated load resistance value input in the control unit 600 is equal to the current load resistance value, it is determined according to the Ohm's low I=V/R that the value of current is constant and the instantaneous amount of drug delivered is constant.

If the load resistance between the iontophoresis electrodes 110 during the iontophoresis procedure is calculated as described, a drug delivery state such as a change in the instantaneous amount of drug delivered can be monitored.

In addition, in the first monitoring step (S100), when the current load resistance value increases to exceed the predetermined range or a change in the load resistance value frequently occurs, it can be determined that the contact state between the subject's skin 900 and the electrode unit 100 and the drug delivery are poor.

Thus, the first monitoring step (S100) compares the current load resistance value with the initially calculated load resistance value input in the control unit 600 in order to stably deliver the amount of drug input, so that it can determine the drug delivery state even during the iontophoresis procedure by measuring the instantaneous amount of drug delivered. According to the drug delivery state, the programmable current unit 300 can be controlled to automatically control the instantaneous amount of drug delivered, and the instantaneous amount of drug delivered can be remote-controlled using the external device 800.

The iontophoretic drug delivery method according to the present invention may comprise a second monitoring step (S200).

The second monitoring step (S200) is a step of supplying alternating current to the iontophoresis electrodes 110 to thereby monitor the drug delivery state based on the measured resistivity value between the tissue resistivity measurement unit 120.

Specifically, the control unit 600 blocks the supply of current from the programmable current unit 300 to the iontophoresis electrodes 110, and controls the impedance detection unit 400 so that the impedance detection unit 400 calculates the tissue resistivity value between the tissue resistivity measurement electrodes 120.

At this time, the alternating current generating unit 410 of the impedance detection unit 400 supplies a high-frequency current of 16 Hz between the tissue resistivity measurement electrodes 120, and the voltage sensor unit 420 senses the voltage between the iontophoresis electrodes 110 to calculate the tissue resistivity value and transmits the calculated tissue resistivity value to the control unit 600.

Furthermore, the control unit 600 determines a target tissue resistivity value according to a conversion equation that coverts the drug delivery amount input in the control unit 600 into a tissue resistivity value. When the target tissue resistivity value and the current tissue resistivity value are compared with each other and the current tissue resistivity value is greater than a predetermined reference value compared to the target tissue resistivity value, the control unit 600 determines that the amount of drug currently accumulated in the skin is lower than the drug delivery amount input in the control unit 600.

On the other hand, the control unit 600 determines a target tissue resistivity value according to a conversion equation that coverts the drug delivery amount input in the control unit 600 into a tissue resistivity value. When the current tissue resistivity value is equal to the target tissue resistivity value or lower than the predetermined reference value, the control unit 600 determines that the amount of drug delivered is greater than the drug delivery amount input in the control unit 600.

As described above, the second monitoring step (S200) compares the current issue resistivity value with the target tissue resistivity value input in the control unit 600 in order to stably deliver the drug. Thus, even during the iontophoresis procedure, the instantaneous amount of drug can be remote-controlled by automatically measuring the amount of drug accumulated in the skin and automatically controlling the programmable current unit 300 from the relationship between the amount of drug delivered to the subject and the drug delivery amount input in the control unit 600. At this time, the instantaneous amount of drug can also be remote-controlled using the external device 800.

Meanwhile, the first monitoring step (S100) or the second monitoring step (S200) may further comprise a step of sending an abnormal drug delivery state to the outside.

Specifically, when the amount of drug delivered is out of the predetermined range of the initial load resistance value or the target tissue resistivity value, the supply of current to the iontophoresis electrodes 110 can be blocked, and the abnormal drug delivery state can be sent to the outside through a warning means, whereby an observer who observes the iontophoresis procedure can rapidly recognize the abnormal drug delivery state and can check whether the drug is stably delivered, through the external device 800, thereby preventing an accident from being caused by mis-operation of the iontophoresis apparatus.

Herein, the warning means may be an external device 800 comprising a display, which switches-off LEDs disposed in the iontophoretic drug delivery apparatus or sounds a warning through a speaker installed in the iontophoretic drug delivery apparatus or communicates with the iontophoretic drug delivery apparatus in a wireless manner. The external device 800 can give the observer a warning of the abnormal state of the iontophoresis procedure.

The iontophoretic drug delivery method according to the present invention may comprise step (S300) of controlling drug delivery.

Step (S300) of controlling drug delivery controls drug delivery based the current load resistance value or tissue resistivity value based on the first monitoring step (S100) and the second monitoring step (S200).

Specifically, when the measured value is out of the predetermined range of the initial load resistance value or target tissue resistivity value in the first monitoring step (S100) or the second monitoring step (S200), the amplitude of current that is supplied to the iontophoresis electrodes 110 can be controlled or the supply of the current can be blocked, thereby controlling the amount of drug delivered or blocking delivery of the drug.

For example, when the first monitoring step (S100) determines that the instantaneous amount of drug delivered decreases, one or more value of the amplitude and duty cycle of the current that is supplied from the programmable current unit 300 can be increased or the frequency of the current can be decreased, in order to increase the instantaneous amount of drug delivered to the skin 900 per unit time, whereby the drug can be stably delivered into the subject's skin 900 in the amount input in the control unit 600.

On the other hand, when the first monitoring step (S100) determines that the instantaneous amount of drug administered increases, one or more value of the amplitude and duty cycle of the current that is supplied from the programmable current unit 300 can be decreased or the frequency of the current can be increased, in order to decrease the instantaneous amount of drug delivered to the skin 900 per unit time, whereby the drug can be stably delivered into the subject's skin 900 in the amount input in the control unit 600.

Meanwhile, when the second monitoring step (S200) determines that the amount of drug accumulated is smaller than the drug delivery amount input in the control unit 600, the amplitude of current that is supplied from the programmable current unit 300 can be maintained or increased to increase the instantaneous amount of drug administered, so that the amount of drug accumulated will be greater than the drug delivery amount input in the control unit 600, whereby the drug can be stably delivered into the subject's skin 900 in the amount input in the control unit 600.

On the other hand, when the second monitoring step (S200) determines that the amount of drug accumulated is greater than the drug delivery amount input in the control unit 600, the current that is supplied from the programmable current unit 300 can be blocked to end the iontophoresis procedure, whereby the drug can be stably delivered into the subject's skin 900 in the amount input in the control unit 600.

In addition, step (S300) of controlling drug delivery may further comprise a step of stopping drug delivery.

In the step of stopping drug delivery, when the temperature measured by the temperature sensor unit 500 is out of a predetermined temperature range, the supply of current to the iontophoresis electrodes 10 can be blocked to stop drug delivery.

Specifically, in order to prevent the subject's skin 900 from getting burned due to an increase in the electrode temperature, when the electrode temperature value provided from the temperature sensor unit 500 is out of the predetermined temperature range input in the control unit 600, the control unit 600 can block the current that is supplied from the programmable current unit 300, thereby stopping drug delivery.

Therefore, the iontophoretic drug delivery apparatus and drug delivery method according to the embodiment of the present invention comprise the iontophoresis electrodes 110 and the tissue resistivity measurement electrodes 120. Thus, it is possible to accurately monitor the state information of the iontophoresis procedure based on the load resistance value and the tissue resistivity value through the impedance detection unit 400. Based on this state information, the drug can be delivered, whereby the drug can be uniformly and stably delivered.

Furthermore, the iontophoretic drug delivery apparatus comprises the temperature sensor unit 500 that measures a portion of a subject into which a drug is delivered. Thus, it is possible to prevent the subject from getting burned due to heating of the electrode unit 100.

In addition, the iontophoretic drug delivery apparatus comprises the wireless communication unit 700 that communicates with the external device 800 in a wireless manner. Thus, the state information of the iontophoresis procedure can be easily checked. In addition, even at long distance, the state information of the iontophoresis procedure can be checked and the iontophoresis procedure can be controlled.

Although the embodiments of the present invention have been described, the scope of the present invention is not limited thereto, and those skilled in the art will appreciate that various modifications, additions and substitutions are possible, without departing from the scope and spirit of the invention as disclosed in the accompanying claims.

### INDUSTRIAL APPLICABILITY

The present invention can be used in health-related industrial fields, including the health care field and the medical device field.

1. An iontophoretic drug delivery apparatus, comprising:
an electrode unit comprising a plurality of iontophoresis electrodes and a plurality of tissue resistivity measurement electrodes;
a programmable current unit configured to control a current that is supplied to the iontophoresis electrodes to thereby control the amount of drug delivered;
an impedance detection unit comprising a detection mode that selectively measures a load resistance value between the iontophoresis electrodes or a tissue resistivity value between the tissue resistivity measurement electrodes so as to monitor the amount of drug delivered; and
a control unit configured to control the programmable current unit by determining the amount of drug delivered or whether the drug is to be delivered, based on the load resistance or tissue resistivity value measured in the impedance detection unit.

2. The iontophoretic drug delivery apparatus of claim 1, comprising a wireless communication unit configured to convert information of the control unit to a wireless signal and communicate the converted wireless signal with an external device in a wireless manner.

3. The iontophoretic drug delivery apparatus of claim 1, wherein the impedance detection unit comprises:
an alternating current generating unit configured to supply alternating current to the iontophoresis electrodes; and
a voltage sensor unit configured to measure a voltage that is generated in the iontophoresis electrodes and the tissue resistivity measurement electrodes.

4. The iontophoretic drug delivery apparatus of claim 1, wherein when the detection mode of the impedance detection unit is an instantaneous drug delivery amount-detecting mode that measures the load resistance value, the control unit supplies alternating current to the iontophoresis electrodes in a state in which operation of the programmable current unit is stopped, and the control unit may control the programmable control unit based on the measured load resistance value between the iontophoresis electrodes.

5. The iontophoretic drug delivery apparatus of claim 1, wherein when the detection mode of the impedance detection mode is a cumulative drug amount-detecting mode that measures the tissue resistivity value, the control unit supplies alternating current to the iontophoresis electrodes in a state in which operation of the programmable current unit is stopped, and the control unit may control the programmable current unit based on the measured tissue resistivity value between the tissue resistivity measurement electrodes so that the drug may be delivered.

6. The iontophoretic drug delivery apparatus of claim 1, wherein the detection mode of the impedance detection unit comprises different detection modes that are sequentially selected and operated for a predetermined time.

7. (canceled)

8. The iontophoretic drug delivery apparatus of claim 2, wherein the external device comprises a display that communicates with the wireless communication unit in a wireless manner to observe information about the subject.

9. The iontophoretic drug delivery apparatus of claim 2, wherein the external device comprises an input means that inputs drug delivery information so as to control the control unit by communication with the wireless communication unit.

10. The iontophoretic drug delivery apparatus of claim 1, wherein the electrode unit comprises an electrode switching unit that, according to the detection mode, selectively connects the iontophoresis electrodes and the tissue resistivity measurement electrodes to the impedance detection unit or selectively changes the polarity of the current that is transferred from the programmable current unit to the iontophoresis electrodes.

11. The iontophoretic drug delivery apparatus of claim 1, wherein the electrode unit is configured such that a pair of the tissue resistivity measurement electrodes disposed between a pair of the iontophoresis electrodes, or a pair of the iontophoresis electrodes disposed between a pair of the tissue resistivity measurement electrodes.

12. The iontophoretic drug delivery apparatus of claim 1, wherein the electrode unit is configured such that the plurality of iontophoresis electrodes and the plurality of tissue resistivity measurement electrodes, which are alternately disposed to be spaced apart from each other, and either the tissue resistivity measurement electrodes disposed between the iontophoresis electrodes, or the iontophoresis electrodes disposed between the tissue resistivity measurement electrodes, are provided in a pair.

13. The iontophoretic drug delivery apparatus of claim 1, wherein the electrode unit is configured such that the iontophoresis electrodes and the tissue resistivity measurement electrodes are radially disposed in concentric circles having different diameters.

14. The iontophoretic drug delivery apparatus of claim 1, wherein the electrode unit further comprises a temperature sensor unit for measuring a temperature of a portion to which the drug is delivered.

15. The iontophoretic drug delivery apparatus of claim 1, wherein the control unit stops operation of the programmable current unit to stop delivery of the drug, when the temperature measured in the temperature sensor unit is out of a predetermined temperature range input in the control unit.

16. The iontophoretic drug delivery apparatus of claim 1, comprising a drug-containing drug pad which is detachably coupled to the iontophoresis electrodes.

17. The iontophoretic drug delivery apparatus of claim 16, wherein the drug pad comprises a portion formed of a porous material so that the drug is impregnated into the portion.

18. The iontophoretic drug delivery apparatus of claim 16, wherein the drug pad comprises an adhesive layer provided on one or both surfaces of the drug pad.

19. The iontophoretic drug delivery apparatus of claim 16, wherein the drug pad comprises a drug information memory unit configured to store information about the drug and provide the information to the control unit.

20. An iontophoretic drug delivery method using an iontophoretic drug delivery apparatus comprising a plurality of iontophoresis electrodes and a plurality of tissue resistivity measurement electrodes, the method comprising:
a first monitoring step of supplying alternating current to the iontophoresis electrodes to thereby monitor a delivery state of the drug based on a measured load resistance value between the iontophoresis electrodes;
a second monitoring step of supplying alternating current to the iontophoresis electrodes to thereby monitor the delivery state of the drug based on a measured tissue resistivity value between the tissue resistivity measurement electrodes; and
a step of controlling delivery of the drug based on the load resistance value or tissue resistivity value measured in the first monitoring step and the second monitoring step.

21. The iontophoretic drug delivery method of claim 20, wherein the iontophoretic drug delivery apparatus further comprise a temperature measurement sensor for measuring the temperature of a portion to which the drug is delivered, and
the step of controlling delivery of the drug further comprises a step of stopping the delivery of the drug by blocking supply of the current to the iontophoresis electrodes when the temperature measured in the temperature measurement sensor is out of a predetermined temperature range.

22. The iontophoretic drug delivery method of claim 20, wherein the first monitoring step comprises blocking the current that is supplied to the iontophoresis electrodes, and supplying a low-frequency alternating current to the iontophoresis electrode, and sensing a voltage between the iontophoresis electrodes to thereby measure the load resistance value.

23. The iontophoretic drug delivery method of claim 20, wherein the second monitoring step comprises blocking the current that is supplied to the iontophoresis electrodes, and supplying a high-frequency alternating current to the iontophoresis electrode.

24. The iontophoretic drug delivery method of claim 20, wherein the first monitoring step or the second monitoring step further comprises a step of blocking the current that is supplied to the iontophoresis electrodes when a current load resistance value or tissue resistivity value is out of a predetermined ideal value range, and sensing the abnormal state or end-state of drug delivery through a warning means.

25. The iontophoretic drug delivery method of claim 20, wherein the first monitoring step or the second monitoring step comprises controlling one or more values selected from among amplitude, frequency and duty cycle values of the current that is supplied to the iontophoresis electrodes, when the current load resistance value or tissue resistivity value is out of a predetermined ideal value range.

26. The iontophoretic drug delivery method of claim 20, wherein the first monitoring step or the second monitoring step comprises controlling one or more values selected from among amplitude, frequency and duty cycle values of the current that is supplied to the iontophoresis electrodes, when a current load resistance value or tissue resistivity value is within a predetermined range.

27. An iontophoretic drug delivery apparatus comprising:
an electrode unit comprising a plurality of iontophoresis electrodes and a plurality of tissue resistivity measurement electrodes spaced apart from the iontophoresis electrodes;
a programmable current unit configured to control an amplitude of a current that is supplied to the iontophoresis electrodes to thereby control an amount of drug delivered;
an impedance detection unit configured to selectively measure either a load resistance between the iontophoresis electrodes, obtained after supplying the current between the iontophoresis electrodes and sensing a voltage, generated due to the load resistance between the iontophoresis electrodes, by the iontophoresis electrodes, or a tissue resistivity, obtained after supplying the current between the iontophoresis electrodes and sensing a voltage, generated due to the tissue resistivity between the tissue resistivity measurement electrodes, by the tissue resistivity measurement electrodes; a control unit configured to control the programmable current unit by determining the amount of drug delivered or whether the drug is to be delivered, based on the load resistance or tissue resistivity value measured in the impedance detection unit; and
a wireless communication unit configured to convert information of the control unit to a wireless signal and communicate the converted wireless signal with an external device.

28. An iontophoretic drug delivery apparatus comprising a plurality of iontophoresis electrodes and a programmable current unit configured to control one or more values selected from among amplitude, frequency and duty cycle values of a current that is supplied to the iontophoresis electrodes to thereby control the amount of drug delivered, the apparatus being configured to:
supply current to the iontophoresis electrodes coming into contact with a body portion;
sense a voltage, generated due to tissue load of the applied current, by tissue resistivity measurement electrodes physically spaced apart from the iontophoresis electrodes, thereby measuring a tissue resistivity; and
monitor a cumulative amount of drug delivered, based on the tissue resistivity, thereby controlling the programmable current.
